(19) United States
(12) Patent Application Publication
(10) Pub. No.: US 2008/0300458 A1
(43) Pub. Date: Dec. 4, 2008

(54) Capsule Type Endoscope Control System

(76) Inventors: Byung Kyu Kim, Seoul (KR); Jong Oh Park, Seoul (KR); Yeh Sun Hong, Seoul (KR)

Correspondence Address: SCHWEITZER CORNMAN GROSS & BONDELL LLP, 292 MADISON AVENUE - 19th FLOOR, NEW YORK, NY, 10017, US

(21) Appl. No.: 11/630,183

(22) PCT Filed: Jun. 21, 2005
(86) PCT No.: PCT/KR05/01915

§ 371 Date: Oct. 18, 2007

(30) Foreign Application Priority Data
Jun. 21, 2004 (KR) 10-2004-0046202

(51) Int. Cl. A61B 1/00

(52) U.S. Cl. 600/118

(57) ABSTRACT

Disclosed is a capsule type endoscope control system which can move to any position, rotate or stop the capsule type endoscope in a human body by a remote control system outside the human body. There is provided a capsule type endoscope control system comprising: a medical capsule equipped with at least one permanent magnet, comprising a wireless transmission circuit for transmitting a series of signals to outside of the body; 2-DOF rotary joint unit for rotating an external permanent magnet in at least two directions, the external permanent magnet applying magnetic magnet forces to the permanent magnets provided in the capsule; a distance sensor for measuring a distance between the external permanent magnet and a surface of the human body; a cartesian coordinate robot for moving the external permanent magnet; a bed for supporting the human body, the bed being able to roll within a certain degree; and a remote control unit outside the human body for controlling operations of the 2-DOF rotary joint unit, the bed and the cartesian coordinate robot.

### TECHNICAL FIELD

The present invention relates to a capsule type endoscope, and more particularly to a capsule type endoscope control system which can move to any position, rotate or stop the capsule type endoscope in a human body by a remote control system outside the human body, by moving and rotating an external permanent magnet which applies magnetic force to the capsule, with a cartesian coordinate robot having a 2-degree of freedom (DOF) rotary joint unit.

### BACKGROUND ART

In general, an endoscope is a general term of medical devices used to diagnose lesions of inner surfaces of hollow organs (e.g., a stomach, an esophagus and etc.), a thoracic cavity and an abdominal cavity, etc. in a human body without a surgical operation. Since the endoscope causes great distress and uncomfortableness to a patient when the endoscope is used, patients do not like the endoscope. For example, in a case of a large intestine endoscope, since the large intestine is bended at a large angle, a pain applied to a patient and a judgment possibility of a lesion are highly affected by experiences and skills of a doctor.

To improve the above-described problems of the conventional endoscope, a virtual colonoscopy or gene test has been suggested. However, these methods are evaluated as indirect methods since a doctor cannot perform biopsy or directly treat affected parts.

In recent years, a swallowable capsule type endoscope equipped with a wireless camera system has been developed to widen ranges of medical diagnosis. The capsule type endoscope made it possible to treat organs that had not been observed by the conventional endoscope (e.g., large intestine, small intestine, etc.) by transmitting information on image of walls of the organs to outside. The above capsule type endoscope comprises a CCD camera and a device for wirelessly transmitting image data obtained by the CCD camera.

### DISCLOSURE OF INVENTION

### Technical Problem

However, since the capsule type endoscope is moved passively depending on peristaltic movements of the organ in the human body, there are drawbacks that it is impossible to freely stop the capsule at a place where detailed observation is needed and to return to a place where the capsule already passed by to observe the place again.

Additionally, Nokia company developed an apparatus illustrated in FIG. 1. The apparatus comprises three stator coils 11-1 through 11-3 outside a human body, the three stator coils being positioned separately on three points of the human body. An armature coil is provided in the capsule inside the human body. The capsule 12 rolls depending on currents of the stator coils 11-1 through 11-3. Accordingly, a photographing angle of a CCD camera provided in the capsule 12 can be adjusted. At this time, the stator coils 11-1 through 11-3, which should be provided to outside of the human body, are provided in a frame having a vest shape and a patient wears it. However, this apparatus has also drawbacks that it is impossible to move the capsule 12 in the organ in the opposite direction or to forcibly move the capsule to a wanted part with promptitude, as with the other conventional apparatuses, since the capsule 12 is also passively moved by peristaltic movements of the organs.

To solve the disadvantages, the applicant (Korea Institute of Science and Technology) filed a patent application (Korean patent application No. 10-2003-0039199) disclosing an apparatus capable of forcibly moving a capsule type endoscope in a human body in a non-contact manner from outside of the human body. Specifically, as shown in FIG. 2, the Korean patent application No. 10-2003-0039199 suggests 5-DOF manipulating apparatus that freely moves or stops the capsule type endoscope in a human body with magnetism of a separate external permanent magnet outside the human body, the capsule type endoscope being equipped with a permanent magnet (or electromagnet).

In other words, according to the Korean patent application No. 10-2003-0039199, it is possible for the external permanent magnet to induce movements of the capsule type endoscope according to magnetization directions of the permanent magnet provided in the capsule type endoscope as illustrated in FIGS. 3 through 9. The apparatus for moving the capsule type endoscope according to the Korean patent application No. 10-2003-0039199 has 5-DOF, i.e., two rotational DOF for rotating the external permanent magnet in two different directions with two center axes, and three linear DOF for moving the external permanent magnet to transverse, longitudinal and vertical directions of the human body.

According to the Korean patent application No. 10-2003-0039199, a distance between the capsule type endoscope and the external permanent magnet is controlled manually. Accordingly, when the capsule type endoscope and the permanent magnet become too close due to an operator's error, the magnetic force becomes too great, so that the capsule type endoscope strongly pushes out a wall of an organ and thus the wall of the organ can be damaged. In contrast, when the capsule type endoscope and the external permanent magnet become distant, the magnetic force between the capsule type endoscope and the external permanent magnet becomes quickly weak and thus the capsule can be missed.

In addition, when the magnetic force is controlled manually with a position of the capsule type endoscope unknown, it is difficult to move the capsule type endoscope smoothly. Further, since the operator should continuously manipulate and maintain the position and direction of the external permanent magnet, she or he can easily feel fatigue.

### Technical Solution

Accordingly, the present invention has been made to solve the above-mentioned problems occurring in the prior art. The object of the present invention is to provide a capsule type endoscope control system which can move to any position, rotate or stop the capsule type endoscope (“the capsule”) in a human body by a remote control system outside the human body, by moving and rotating an external permanent magnet which applies magnetic force to the capsule, with a cartesian coordinate robot having a 2-DOF rotary joint unit.

Another object of the present invention is to control an excessive magnetic force not to be applied to the capsule in the human body and to prevent inner wall of digestive organs in the human body from being damaged due to the excessive magnetic force, when moving to any position, rotating or stopping a capsule in the human body, by controlling the external permanent magnet using the cartesian coordinate robot having a 2-DOF rotary joint unit.

A still another object of the present invention is to reduce stick-slip phenomenon and to allow a joystick outside the human body to control movements of the capsule in the human body, by making a capsule roll, yaw or pitch continuously when moving forward the capsule and adjusting a forward direction of a joystick to the forward direction of the capsule through sensing the forward direction of the capsule. Further, the object of the present invention is to make it possible to diagnose or treat digestive organs with softness, safety and comfortableness and to move the capsule precisely, by providing functions of measuring a distance from the human body surface to the capsule.

In order to accomplish the object, there is provided a capsule type endoscope control system for diagnosing digestive organs in a human body comprising: a medical capsule equipped with at least one permanent magnet, Hall sensors and a camera such as CCD camera to diagnose the digestive organs, comprising a wireless transmission circuit for transmitting a series of signals to outside of the body; 2-degree of freedom (DOF) rotary joint unit for rotating an external permanent magnet in at least two directions, the external permanent magnet applying magnetic forces to the permanent magnets provided in the capsule; a distance sensor attached to a lower end of the 2-DOF rotary joint unit, for measuring a distance between the external permanent magnet and a surface of the human body; a cartesian coordinate robot for moving the external permanent magnet and the 2-DOF rotary joint unit; a bed for supporting the human body, the bed being able to roll within a certain degree; and a remote control unit outside the human body for controlling operations of the 2-DOF rotary joint unit, the bed and the cartesian coordinate robot, thereby moving to any position, rotating or stopping the capsule in the human body.

Preferably, the Hall sensors provided in the capsule may provide information on a magnetic force applied from the external permanent magnet to the capsule and a distance between the capsule and the external permanent magnet, and Hall sensor signals may be transmitted to the remote control unit via the wireless transmission circuit, together with an image signal, the image being obtained by the camera.

Preferably, the 2-DOF rotary joint unit may comprise a plurality of joint driving motors for driving the 2-DOF rotary joint unit, and wherein the 2-DOF rotary joint unit may make the capsule in the human body roll, yaw or pitch by rotating the external permanent magnet in at least two directions according to the remote control unit's control of the 2-DOF rotary joint unit's rotation angle, the external permanent magnet being attached to the lower end of the 2-DOF rotary joint unit.

Preferably, the cartesian coordinate robot may comprise a plurality of robot driving motors for driving the cartesian coordinate robot, and the cartesian coordinate robot may move the external permanent magnet to a transverse direction, a longitudinal direction and a vertical direction of the human body according to the remote control unit's control of the cartesian coordinate robot's speed and displacement.

Preferably, the bed may comprise bed driving motors for driving the bed to roll and the bed may roll around longitudinal axis of the bed according to the remote control unit's control of the bed's angle.

Preferably, the remote control unit may comprise: a signal receiver for receiving an image signal and Hall sensor signals transmitted from the wireless transmission circuit of the capsule in the human body, the image being obtained by the camera; a joystick for outputting a command signal controlling the robot driving motors for controlling speed and displacement of the cartesian coordinate robot, a command signal controlling the joint driving motor for controlling rotation angle of the 2-DOF rotary joint unit, and a command signal controlling the bed driving motors for controlling angle of the bed by using a bed adjustment switch, according to an operator's operation; a main controller for receiving the image signal from the signal receiver, for displaying the image on a screen, for generating driving motor control signal for the cartesian coordinate robot and 2-DOF rotary joint unit by combining the command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic force applied to the capsule constant by analyzing the Hall sensor signals of the capsule, for calculating a distance between a surface of the human body and the capsule using the Hall sensor signals and a distance obtained by the distance sensor, and for displaying the calculated distance on the screen; a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body; a 2-DOF joint unit controller for controlling the 2-DOF joint unit to adjust rotation angle of the 2-DOF joint unit according to the driving motor control signal outputted from the main controller or outputted as a result of manual operations to rotate the external permanent magnet in at least two directions, thereby making the capsule in the human body roll, pitch or yaw; and a bed rotation controller for driving a bed driving motor provided in the bed according to the signal that controls the bed's angle to roll around longitudinal axis of the bed, the signal being outputted from the bed adjustment switch provided in the joystick.

Preferably, the main controller may recognize a shape change of the digestive organs using a frame grabber function from the image obtained by the camera, determine and estimate a forward direction of the capsule in the human body using the camera image or the signals of the two Hall sensors provided in the capsule, and display a position and a path of the capsule in the human body against a fixed coordinate outside the human body by considering the image signal and Hall sensor signals transmitted from the capsule, the position of the capsule against the fixed coordinate, rotation angle of the external permanent magnet, a distance between the capsule and the external permanent magnet, and the estimated direction of the capsule.

Preferably, the main controller may estimate the distance between the external permanent magnet and the capsule by analyzing the Hall sensor signals, measure the distance between the external permanent magnet and the body surface using the distance sensor and thus calculate the distance from the body surface to the capsule.

Preferably, the main controller may further comprise: a robot control signal outputting unit for outputting control signal to control speed of the cartesian coordinate robot in X and Y axes direction by combining the command signal controlling the robot driving motors, direction of the capsule and coordinate of the capsule, the command signal controlling speed of the cartesian coordinate robot in X and Y axes direction, and outputting control signal to control speed and displacement of the cartesian coordinate robot in Z axis direction by using magnetic force information obtained by combining the command signal controlling the robot driving motors, measured magnetic force of the capsule and reference input value of magnetic force, the command signal controlling speed and displacement of the cartesian coordinate robot in Z axis direction; and a direction determining and coordinate calculating unit for determining direction of the capsule by analyzing the two Hall sensor signals transmitted from the signal receiver and the information of shape change recognized by a frame grabber function unit, calculating the coordinate value of the capsule and transmitting the coordinate value to the robot control signal outputting unit and 2-DOF joint unit controller.

Preferably, the main controller may further comprise: a magnetic force measuring unit for measuring a magnetic force applied to the capsule by analyzing the Hall sensor signals transmitted from the signal receiver and for transmitting the measured value of the magnetic force to the robot control signal outputting unit; a permanent magnet distance estimating unit for estimating a distance between the permanent magnets of the capsule and the external permanent magnet by analyzing the Hall sensor signals transmitted from the signal receiver; and a capsule depth calculating unit for calculating a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.

Preferably, the camera may be a CCD camera.

Preferably, the distance sensor may be a photoelectric sensor or ultrasonic sensor.

Alternatively, there is provided a capsule type endoscope control system for diagnosing digestive organs in a human body comprising: a medical capsule equipped with at least one permanent magnet, Hall sensors and a camera to diagnose the digestive organs, comprising a wireless transmission circuit for transmitting a series of signals to outside of the body; multi-degree of freedom (DOF) rotary joint unit for rotating an external permanent magnet in at least two directions, the external permanent magnet applying magnetic forces to the permanent magnets provided in the capsule; a distance sensor attached to a lower end of the multi-DOF rotary joint unit, for measuring a distance between the external permanent magnet and a surface of the human body; a cartesian coordinate robot for moving the external permanent magnet and the multi-DOF rotary joint unit; a bed for supporting the human body, the bed being able to roll within a certain degree; and a remote control unit outside the human body for controlling operations of the multi-DOF rotary joint unit, the bed and the cartesian coordinate robot, thereby moving to any position, rotating or stopping the capsule in the human body.

Alternatively, there is provided a capsule type endoscope control system for diagnosing and/or treating digestive organs in a human body comprising: a medical capsule equipped with at least one permanent magnet, Hall sensors, a medicine supplying unit and a camera to diagnose and/or treat the digestive organs, comprising a wireless transmission circuit for transmitting a series of signals to outside of the body; multi-degree of freedom (DOF) rotary joint unit for rotating an external permanent magnet in at least two directions, the external permanent magnet applying magnetic forces to the permanent magnets provided in the capsule; a distance sensor attached to a lower end of the multi-DOF rotary joint unit, for measuring a distance between the external permanent magnet and a surface of the human body; a cartesian coordinate robot for moving the external permanent magnet and the multi-DOF rotary joint unit; a bed for supporting the human body, the bed being able to roll within a certain degree; and a remote control unit outside the human body for controlling operations of the multi-DOF rotary joint unit, the bed and the cartesian coordinate robot, thereby moving to any position, rotating or stopping the capsule in the human body.

### Advantageous Effects

According to the present invention, when moving to any position, rotating or stopping the capsule in the human body, the external permanent magnet outside the human body is controlled by the cartesian coordinate robot having 2-DOF rotary joint unit, so that it is possible to control an excessive magnetic force not to be applied to the capsule in the human body. Accordingly, it is possible to prevent inner walls of the digestive organs in the human body from being damaged due to the excessive magnetic force.

In addition, according to the present invention, a repetitive dither movement such as rolling, yawing or pitching movement is applied to the capsule and moving direction of the joystick is adjusted to moving direction of the capsule by sensing the moving direction of the capsule, when moving the capsule in the human body. Accordingly, it is possible to reduce the stick-slip phenomenon and to easily manipulate the movement of the capsule in the human body with the joystick. Further, there is provided a function of measuring a depth of the capsule in the human body (that is, a distance between the capsule and the surface of the human body), so that it is possible to perform a diagnosis or treatment of the digestive organs with softness, safety and ease while correctly controlling the movement of the capsule.

### BRIEF DESCRIPTION OF THE DRAWINGS

The above and other objects, features and advantages of the present invention will be more apparent from the following detailed description taken in conjunction with the accompanying drawings, in which:

FIG. 1 illustrates a capsule type endoscope controlled by external stator coils according to the prior art;

FIG. 2 illustrates a structure of a capsule type endoscope controlling robot according to the prior art;

FIGS. 3 through 9 illustrate movements and rotations of a capsule type endoscope by an external permanent magnet;

FIG. 10 through 12 illustrate detailed configuration of a capsule type endoscope according to an embodiment of the present invention.

FIG. 13 illustrates configuration of a capsule type endoscope control system according to an embodiment of the present invention;

FIG. 14 illustrates that the bed of FIG. 13 inclines to one side;

FIG. 15 illustrates a principle of calculating a distance from a human body surface to the capsule in the human body according to an embodiment of the present invention;

FIG. 16 illustrates detailed configuration of a capsule type endoscope control system according to an embodiment of the present invention;

FIGS. 17 through 19 illustrate a principle of sensing a rotating direction of a capsule when two Hall sensors are attached to a surface of the capsule according to an embodiment of the present invention; and

FIGS. 20 through 22 illustrate exemplary views of a rolling, pitching and yawing movement of the capsule in the human body according to an embodiment of the present invention.

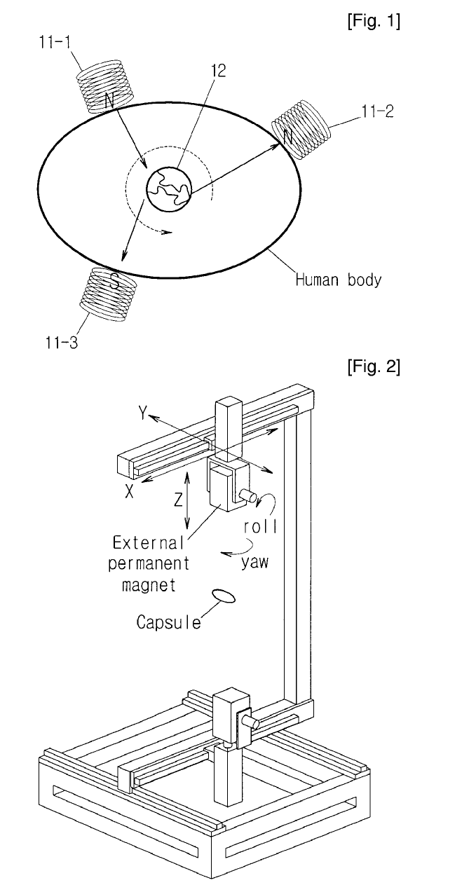

### BEST MODE FOR CARRYING OUT THE INVENTION

Hereinafter, preferred embodiments of the present invention will be described with reference to the accompanying drawings. In the following description of the present invention, detailed description of known functions and configurations incorporated herein will be omitted when it may make the subject matter of the present invention rat her unclear.

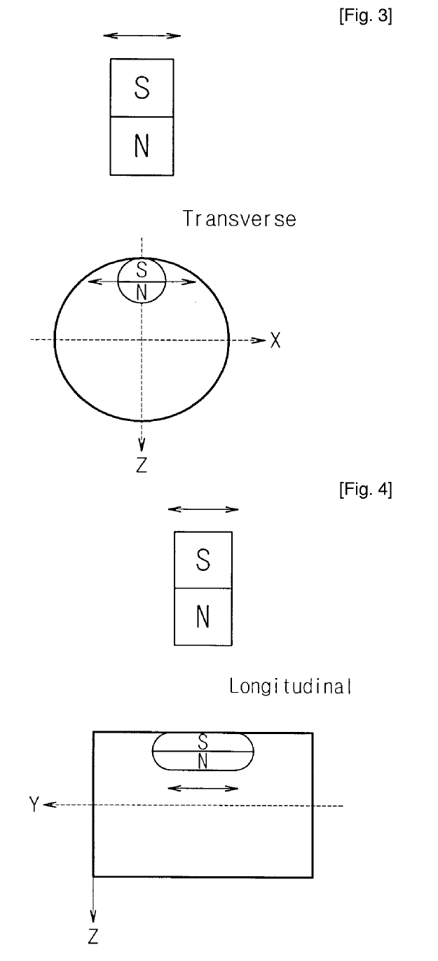

FIGS. 3 through 9 briefly illustrate an external permanent magnet and the capsule type endoscope in the human body. To effectively illustrate movements of the capsule type endoscope, only the permanent magnet is illustrated without any other components of the capsule type endoscope.

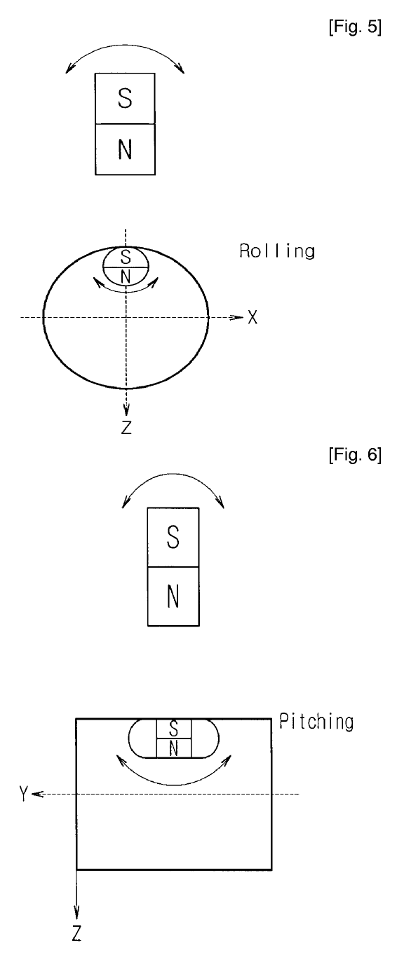

FIGS. 3 through 6 illustrate movements of the capsule type endoscope in case that the longitudinal direction of the external permanent magnet is orthogonal to the longitudinal direction of the capsule type endoscope. FIG. 3 illustrates that the capsule type endoscope moves in a transverse direction of the human organ, as the external permanent magnet moves in a direction parallel with the transverse direction. FIG. 4 illustrates that the capsule type endoscope moves in a longitudinal direction of the human organ, as the external permanent magnet moves in a direction parallel with the longitudinal direction. FIG. 5 illustrates that rolling movement of the external permanent magnet in a certain direction makes the capsule type endoscope roll. FIG. 6 illustrates that rolling movement of the external permanent magnet in another direction makes the capsule type endoscope pitch.

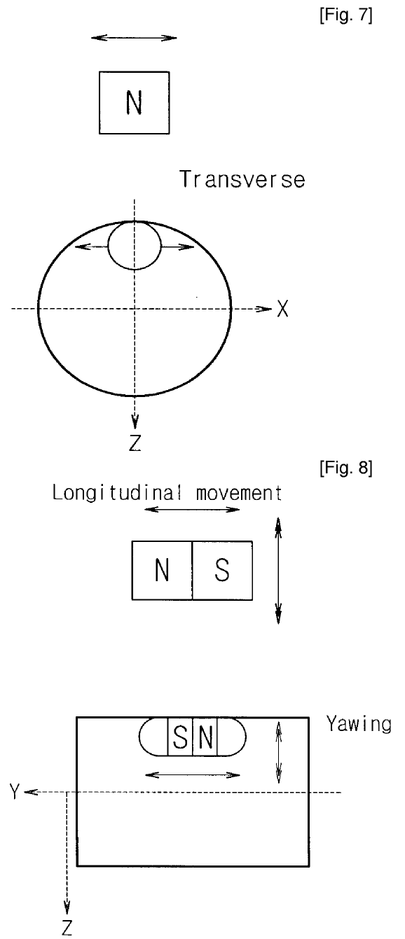

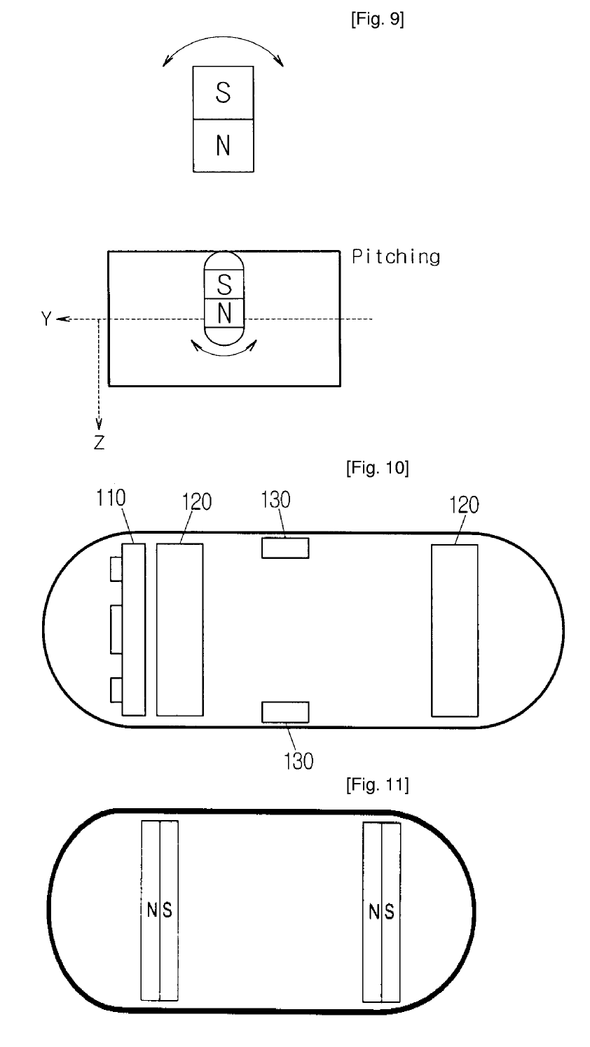

In contrast, FIGS. 7 through 9 illustrate movements of the capsule type endoscope in case that the longitudinal direction of the external magnet is parallel with the longitudinal direction of the capsule type endoscope. FIG. 7 illustrates that the capsule type endoscope moves in a transverse direction of the human organ, as the external permanent magnet moves in a direction parallel with the transverse direction. FIG. 8 illustrates that yawing or longitudinal movements of the external permanent magnet makes the capsule type endoscope yaw or move in longitudinal direction, respectively. FIG. 9 illustrates that rolling movement of the external permanent magnet in a certain direction makes the capsule type endoscope pitch.

The object of the present invention is to implement a remote control system for controlling movements of capsule type endoscope in a human body. For example, the system can control the capsule type endoscope to roll/pitch/yaw, move forward/backward/rightward/leftward, and stop.

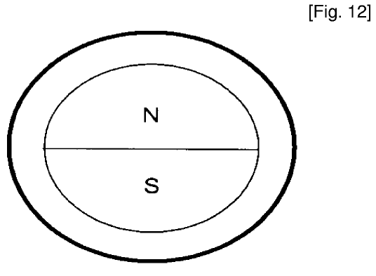

FIG. 10 illustrates exemplary configuration of the capsule type endoscope according to a preferred embodiment of the present invention. The capsule type endoscope comprises: a camera module 110 for taking image of digestive organs; permanent magnets 120 for making the capsule type endoscope move variously, by means of magnetic forces between the permanent magnets 120 and the external permanent magnet outside the human body; and Hall sensors 130 for providing information on a magnetic force applied from the external permanent magnet to the capsule type endoscope and distance between the capsule and the external permanent magnet, each one of the Hall sensors outputting signal having different amplitude according to the rotating direction of the capsule type endoscope. The capsule type endoscope may further comprise: a wireless transmission circuit (not illustrated) for transmitting Hall sensor signals to a remote control unit outside the human body; a battery (not illustrated) for supplying the capsule type endoscope with electric power; and other sensors (not illustrated) for sensing conditions inside the digestive organs such as temperature sensors, pH sensors, pressure sensors and acceleration sensors etc. FIG. 10 illustrates an exemplary view of the capsule type endoscope. Without regard to the capsule type endoscope illustrated in FIG. 10, the capsule type endoscope can be implemented variously. For example, the number of the permanent magnets, the shape of the permanent magnets, etc. can be designed differently depending on operator's purpose. In this regard, FIGS. 11 and 12 illustrate cross-sections of the capsule type endoscope according to preferred embodiments of the present invention.

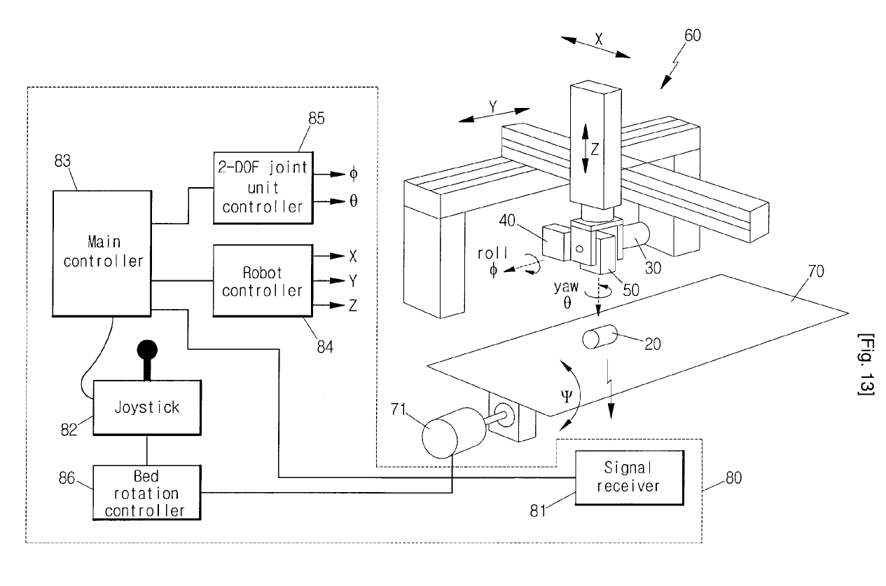

Specifically, as illustrated in FIG. 13, a capsule type endoscope control system according to an embodiment of the present invention comprises a medical capsule 20 equipped with at least one permanent magnet (or electromagnet) and Hall sensors for diagnosing digestive organs of the human body, a 2-DOF rotary joint unit 30 for rotating an external permanent magnet 50 in at least two directions with center axes (roll axis and yaw axis), a distance sensor (such as a photoelectric sensor or an ultrasonic sensor) 40 attached to a lower end of the 2-DOF rotary joint unit 30, a cartesian coordinate robot 60 for moving the external permanent magnet 50 and the 2-DOF rotary joint unit 30, a bed 70 for supporting the human body, the bed being able to roll within a certain degree, and a remote control unit 80 outside the human body for controlling operations of the 2-DOF rotary joint unit 30, the bed 70 and the cartesian coordinate robot 60.

The medical capsule 20 is equipped with at least one permanent magnet which is magnetized in a transverse direction, a camera such as a CCD camera, a lighting device, Hall sensors and a wireless transmission circuit therein. The Hall sensors provide information on a magnetic force applied to the capsule and a distance between the capsule 20 and the external permanent magnet 50. Signals of the Hall sensors are transmitted to the remote control unit 80 outside the human body via the wireless transmission circuit, together with an image signal of the camera.

The 2-DOF rotary joint unit 30 comprises a plurality of joint driving motors for driving the 2-DOF rotary joint unit 30. The 2-DOF rotary joint unit 30 makes the capsule 20 roll, pitch or yaw by rotating the external permanent magnet 50 with an angle (θ) and an angle (Φ) according to remote control unit's control of the 2-DOF rotary joint unit's rotation angle.

The distance sensor 40 is attached to the lower end of 2-DOF rotary joint unit 30 to measure a distance between the external permanent magnet 50 and a surface of the human body according to a non-contact distance measuring method and to transmit a result of the measurement to the remote control unit 80. At this time, the non-contact distance measuring method can use a photoelectric sensor or an ultrasonic sensor.

The cartesian coordinate robot 60 is an electric driving device comprising a plurality of robot driving motors for driving the cartesian coordinate robot 60. The cartesian coordinate robot 60 moves the external permanent magnet 50 to a transverse direction (X), a longitudinal direction (Y) and a vertical direction (Z) of the human body according to the remote control unit's control of the cartesian coordinate robot's speed and displacement.

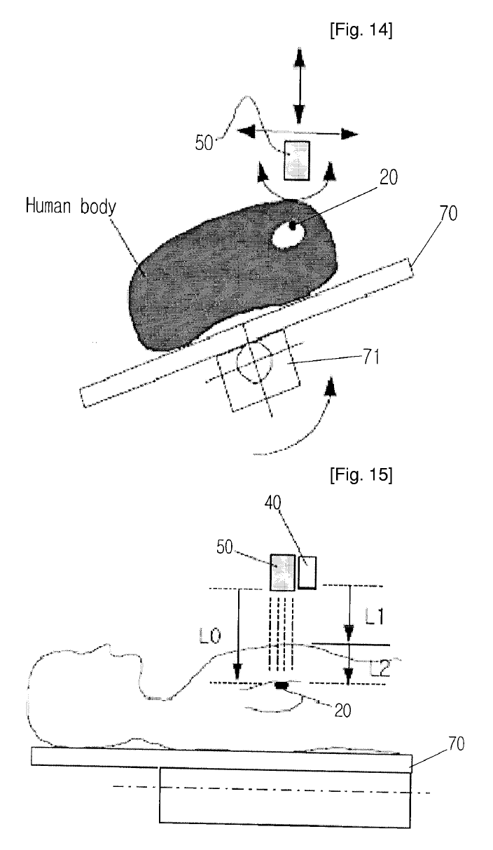

The bed 70 is a table for supporting the human body. The bed is an auxiliary device equipped with bed driving motors 71 for driving the bed to roll, as shown in FIG. 14. The bed can roll around longitudinal axis of the bed (i.e., longitudinal axis of the human body) according to the remote control unit's control of the bed's angle (Ψ) (preferably, within a range of 15 degrees). Thus, the rolling movement of the bed 70 can help the external permanent magnet vertically approach to the side surface of the human body.

The remote control unit 80 controls operations of the robot driving motors for the cartesian coordinate robot 60 and the joint driving motors for the 2-DOF rotary joint unit 30 using joystick operations and stick-slip preventing operations by an operator, receives an image signal from the capsule 20 to display the image on a screen, receives Hall sensor signals from the capsule 20 to control a Z axis displacement of the cartesian coordinate robot 60, and displays a position and a path of the capsule in the human body against a fixed coordinate outside the human body by considering the image signal, the Hall sensor signals, a position against the fixed coordinate, rotation angles (θ,Φ) of the external permanent magnet, a distance between the capsule and the external permanent magnet, and the estimated direction of the capsule.

For performing the above functions, the remote control unit 80 comprises a signal receiver 81, a joystick 82, a main controller 83, a robot controller 84, a 2-DOF joint unit controller 85 and a bed rotation controller 86.

The signal receiver 81 receives the image signal and Hall sensor signals transmitted from the wireless transmission circuit of the capsule 20 and transmits them to the main controller 83.

The joystick 82 outputs a command signal controlling the robot driving motors for controlling speed and displacement of the cartesian coordinate robot, a command signal controlling the joint driving motor for controlling rotation angles (θ,Φ) of the 2-DOF rotary joint unit and a command signal controlling the bed driving motors for controlling angle (Ψ) of the bed by using a bed adjustment switch, according to the operator's operation.

The main controller 83 receives the image signal, the image being photographed by the camera provided in the capsule 20 in the human body, from the signal receiver 81 and displays the image on the screen. The main controller 83 combines the command signals outputted from the joystick and stick-slip preventing operations to generate driving motor control signals for the cartesian coordinate robot 60 and the 2-DOF rotary joint unit 30. Then, the main controller 83 outputs the generated driving motor control signals to the corresponding controllers 84, 85.

The main controller controls a Z axis driving motor to adjust displacement of the cartesian coordinate robot in a Z axis direction to keep the magnetic force applied to the capsule constant by analyzing the Hall sensor signals of the capsule 20. And, the main controller calculates a distance from the body surface to the capsule 20 in the human body using the Hall sensor signals and a distance obtained by the distance sensor and displays the distance from the body surface to the capsule 20 on the screen. In addition, the main controller recognizes a shape change of the digestive organs using a frame grabber function from the image, determines and estimates an forward direction of the capsule 20 in the human body using the camera image or the two Hall sensor signals. Further, the main controller displays a position and a path of the capsule in the human body against a fixed coordinate outside the human body by considering the image signal and Hall sensor signals transmitted from the capsule 20, a position against the fixed coordinate, rotation angles of the external permanent magnet 50, the distance between the capsule 20 and the external permanent magnet 50, and the estimated direction of the capsule 20.

As shown in FIG. 15, the distance from the body surface to the capsule 20 in the human body is calculated as follows. A distance (L0) between the external permanent magnet 50 and the capsule 20 is estimated by analyzing the Hall sensor signals from the capsule 20. And, a distance (L1) between the external permanent magnet and the body surface is measured by the distance sensor 40. Accordingly, the distance (L2) from the body surface to the capsule 20 is calculated.

The robot controller 84 controls X and Y axes driving motors of the cartesian coordinate robot to adjust speed of the cartesian coordinate robot and controls the Z axis driving motor to adjust speed and displacement of the cartesian coordinate robot, according to the driving motor control signal for the cartesian coordinate robot, to move the external permanent magnet in a transverse direction (X), a longitudinal direction (Y) and a vertical direction (Z) of the human body to move the capsule in the human body.

The 2-DOF joint controller 85 controls the 2-DOF joint unit to adjust rotation angles of the 2-DOF joint unit, according to the driving motor control signal outputted from the main controller or outputted as a result of manual operations, to rotate the external permanent magnet with the angle (θ) and the angle (Φ), thereby making the capsule in the human body roll, yaw or pitch. In addition, it is possible to make the capsule move variously or vertically approach to the side surface of the human body by bed's rotation with an angle (Ψ).

The bed rotation controller 86 drives the bed driving motor 71 provided in the bed to rotate the bed 70 around longitudinal axis of the bed with the angle (Ψ) according to the signal that controls the bed's angle (Ψ), the signal outputted from the bed adjustment switch provided in the joystick 82.

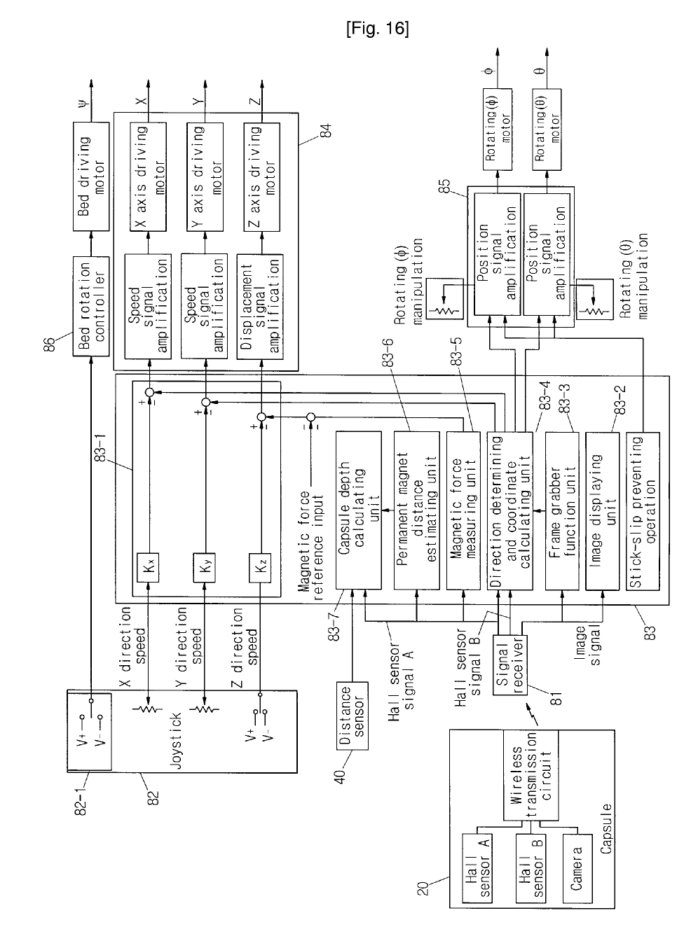

Hereinafter, the main controller 83 described above will be more specifically explained with reference to FIG. 16. The main controller 83 comprises a robot control signal outputting unit 83-1, an image displaying unit 83-2, a direction determining and coordinate calculating unit 83-4, a magnetic force measuring unit 83-5, a permanent magnet distance estimating unit 83-6 and a capsule depth calculating unit 83-7. The robot control signal outputting unit 83-1 outputs control signal to control speed of the cartesian coordinate robot in X and Y axes direction by combining the command signal controlling speed of the cartesian coordinate robot in X and Y axes direction, direction of the capsule and coordinate of the capsule. And, the robot control signal outputting unit 83-1 outputs control signal to control speed and displacement of the cartesian coordinate robot in Z axis direction by using magnetic force information obtained by combining the command signal controlling speed and displacement of the cartesian coordinate robot in Z axis direction, measured magnetic force of the capsule and reference input value of magnetic force.

The image displaying unit 83-2 analyzes the image signal of the capsule 20 in the human body transmitted from the signal receiver 81 and displays the image of the digestive organ on the screen.

The direction determining and coordinate calculating unit 83-4 determines direction of the capsule by analyzing the two Hall sensor signals transmitted from the signal receiver and the information of shape change recognized by a frame grabber function unit, calculates the coordinate value of the capsule and transmits the coordinate value to the robot control signal outputting unit 83-1 and 2-DOF joint unit controller 85.

The magnetic force measuring unit 83-5 measures a magnetic force applied to the capsule by analyzing the Hall sensor signals transmitted from the signal receiver and transmits the measured value of the magnetic force to the robot control signal outputting unit.

The permanent magnet distance estimating unit 83-6 estimates a distance between the permanent magnets of the capsule and the external permanent magnet by analyzing the Hall sensor signals transmitted from the signal receiver 81.

The capsule depth estimating unit 83-7 calculates a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.

With the capsule type endoscope control system according to the present invention having the above-described configuration, as operator inputs speed values for external permanent magnet's movements in a transverse direction and a longitudinal direction of the human body by the operator's manipulation of the joystick, X and Y driving motors of the cartesian coordinate robot 60 are operated by the robot control signal outputting unit 83-1. Thus, the capsule in the human body is moved corresponding to the operations of the X and Y driving motors.

The external permanent magnet 50 is moved in the vertical direction along the Z axis of the cartesian coordinate robot 60. In a manual mode, the external permanent magnet is moved by using information on speed and displacement of the cartesian coordinate robot 60 in Z axis direction, the information being inputted through the joystick operation. And, in an automatic mode, displacement of the external permanent magnet is automatically controlled to keep a distance between the capsule 20 and the external permanent magnet 50 constant, by considering reference input values of magnetic force (They are predetermined values per each digestive organ and can be set by the system operator) aiming at keeping a magnetic force between the external permanent magnet 50 and the permanent magnets in the capsule constant against value of magnetic force measured by the Hall sensor signals from the capsule 20.

In addition, according to the present invention, the main controller 83 of the remote control unit 80 receives the image signal photographed by the camera provided in the capsule 20 via the wireless transmission circuit and displays the image on the screen. In an operating mode, the capsule is moved forward, backward and rotated based on a viewing direction of the camera provided in the capsule. Thus, values inputted by manipulating the joystick need to be transformed into components in a transverse direction (X axis direction) and longitudinal direction (Y axis direction) based on the forward direction of the capsule. For doing so, it is necessary to know a relative angle between the longitudinal axis of the cartesian coordinate robot 60 and the longitudinal axis of the capsule 20 in the human body.

There are methods of finding out the relative angle between the longitudinal axis of the cartesian coordinate robot 60 and the longitudinal axis of the capsule 20 in the human body. In the first place, as shown in FIGS. 3 through 6, the permanent magnets in the capsule are magnetized in a radial direction. As shown in FIG. 5, if the external permanent magnet 50 is rotated, the capsule 20 in the human body rolls corresponding to movements of the external permanent magnet 50. At this time, the external permanent magnet 50 and the capsule 20 rolls in opposite directions. And, if the external permanent magnet 50 rolls with the angle (θ) and the angle (Φ) simultaneously, rotating movement of the capsule 20 is maximized. Accordingly, in order to find out the relative angle between the longitudinal axis of the cartesian coordinate robot and the longitudinal axis of the capsule 20 and adjust the longitudinal axis of the capsule 20 to the longitudinal axis of the cartesian coordinate robot, it is necessary to find out how the image is changed. In this connection, if a rotating direction of the image displayed by the remote control unit 80 is opposite to a rotating direction of the external permanent magnet 50, it can be regarded that the longitudinal axis of the capsule 20 is parallel with the longitudinal axis of the external permanent magnet 50.

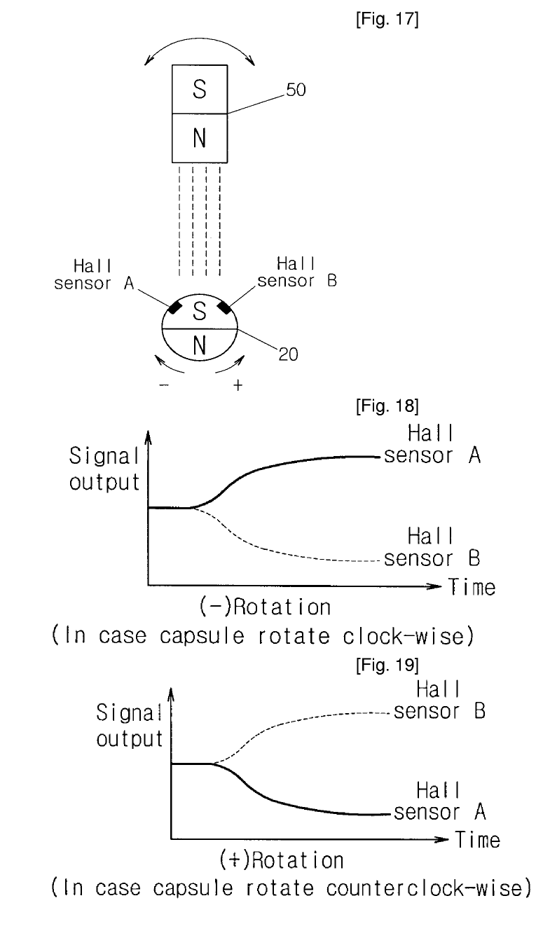

In the second place, as shown in FIG. 17, two Hall sensors are attached to a surface of the capsule 20. In this case, it is possible to find out a rotating direction of the capsule 20 by measuring amplitude of the Hall sensor signals. Further, it is possible to find out the relative angle between a direction of the external permanent magnet's rotation and a direction of the capsule's rotation.

It is possible to find out the relative angle between the longitudinal axis of the cartesian coordinate robot 60 and the longitudinal axis of the capsule 20 in the human body, by measuring the rotation angle (θ, Φ) of the external permanent magnet 50 which the capsule 20 highly responds to the movements of the external permanent magnet and measuring the relative angle between the direction of the external permanent magnet's rotation and the direction of the capsule's rotation according to the above-mentioned methods.

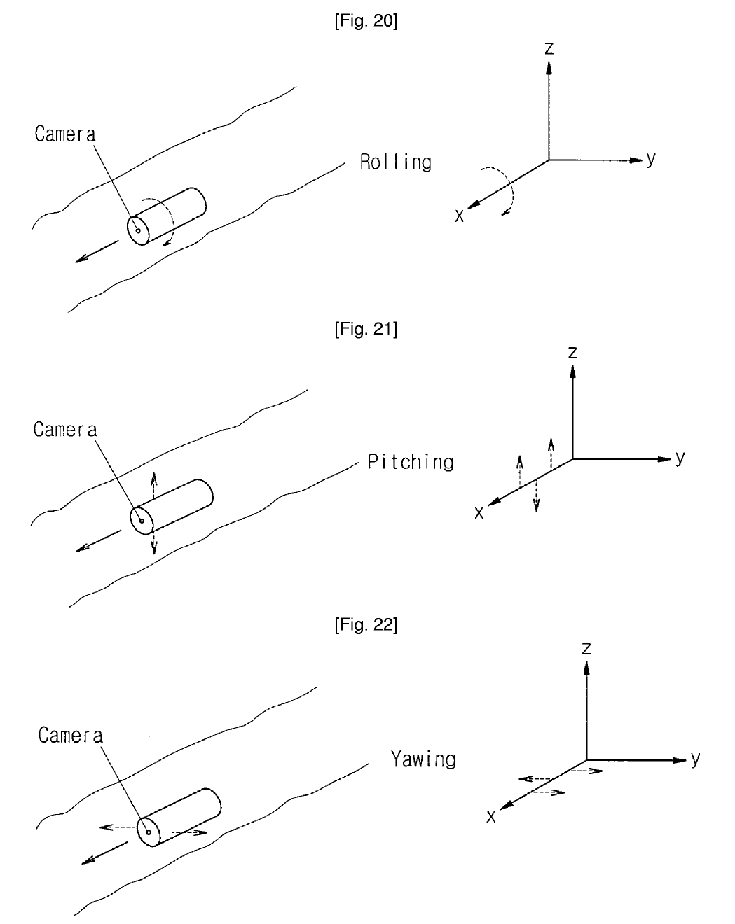

Additionally, according to the present invention, the rotating movements of the external permanent magnet 50 with the angle (θ) and the angle (Φ) can make the capsule 20 in the human body roll, pitch and yaw. For convenience, FIGS. 20 to 22 illustrate the capsule type endoscope simply as a cylinder with a camera. FIG. 20 illustrates that the capsule moves forward with rolling movement. Specifically, if we assume the moving direction of the capsule as “x” axis direction, the capsule is rolling around x axis. FIG. 21 illustrates that the capsule moves forward with pitching movement. Specifically, when moving forward in the “x” axis direction, the capsule experiences dither motion in the “z” axis direction orthogonal to the “x” axis direction. FIG. 22 illustrates that the capsule moves forward with yawing movement. Specifically, when moving forward in the “x” direction, the capsule experiences dither motion in the “y” axis direction. The “x”, “y” and “z” axes mentioned in FIGS. 20 through 22 are introduced here to simply explain rolling, pitching and yawing movements of the capsule in detail. Thus, it is okay not to consider the “x”, “y” and “z” axes to be the “X”, “Y” and “Z” axes of the cartesian coordinate robot. From the above descriptions with reference to FIGS. 20 through 22, it is possible to know that the external permanent magnet's movements with the 2-DOF joint unit can cause various movements of the capsule in the human body. With the various movements of the capsule (i.e. stick-slip prevention movements), it is possible to prevent the stick-slip phenomenon since the capsule 20 is always under dynamic frictional state. Without such movements of the capsule, it is difficult to prevent the stick-slip phenomenon which the capsule repeatedly stops and moves due to a difference between static frictional force and dynamic frictional force.

As described above, according to the present invention, there is provided a capsule type endoscope control system capable of moving a capsule in the human body with magnetic force outside the human body, so that it is possible to move to any position, to rotate or to stop the capsule in the human body through remote control operations outside the human body.

### INDUSTRIAL APPLICABILITY

While the present invention has been shown and described with reference to certain preferred embodiments thereof, it will be understood by those skilled in the art that various changes in form and details may be made therein without departing from the spirit and scope of the present invention as defined by the appended claims. For example, there may be provided a multiple DOF robot having a relatively large operating space, instead of the cartesian coordinate robot and the rotatable bed. In this case, since the function of the 2-DOF rotary joint unit which rotates the external permanent magnet around the yaw axis is overlapped with a DOF of a robot end axis, it is possible to replace it with 1-DOF rotary joint which rotates the external permanent magnet around the roll axis only.

1. A capsule type endoscope control system for diagnosing digestive organs in a human body comprising:
a medical capsule equipped with at least one permanent magnet, Hall sensors and a camera to diagnose the digestive organs, comprising a wireless transmission circuit for transmitting a series of signals to outside of the body;
a 2-degree of freedom (DOF) rotary joint unit for rotating an external permanent magnet in at least two directions, the external permanent magnet applying magnetic forces to the permanent magnets provided in the capsule;
a distance sensor attached to a lower end of the 2-DOF rotary joint unit, for measuring a distance between the external permanent magnet and a surface of the human body;
a cartesian coordinate robot for moving the external permanent magnet and the 2-DOF rotary joint unit;
a bed for supporting the human body, the bed being able to roll within a certain degree; and
a remote control unit outside the human body for controlling operations of the 2-DOF rotary joint unit, the bed and the cartesian coordinate robot, thereby moving to any position, rotating or stopping the capsule in the human body.

2. The control system according to claim 1, wherein the Hall sensors provided in the capsule provides information on a magnetic force applied from the external permanent magnet to the capsule and a distance between the capsule and the external permanent magnet, and Hall sensor signals are transmitted to the remote control unit via the wireless transmission circuit, together with an image signal, the image being obtained by the camera.z

3. The control system according to claim 1, wherein the 2-DOF rotary joint unit comprises a plurality of joint driving motors for driving the 2-DOF rotary joint unit, and wherein the 2-DOF rotary joint unit makes the capsule in the human body roll, yaw or pitch by rotating the external permanent magnet in at least two directions according to the remote control unit's control of the 2-DOF rotary joint unit's rotation angle, the external permanent magnet being attached to the lower end of the 2-DOF rotary joint unit.

4. The control system according to claim 1, wherein the cartesian coordinate robot comprises a plurality of robot driving motors for driving the cartesian coordinate robot, and wherein the cartesian coordinate robot moves the external permanent magnet to a transverse direction, a longitudinal direction and a vertical direction of the human body according to the remote control unit's control of the cartesian coordinate robot's speed and displacement.

5. The control system according to claim 1, wherein the bed comprises bed driving motors for driving the bed to roll and wherein the bed rolls around a longitudinal axis of the bed according to the remote control unit's control of a bed angle.

6. The control system according to claim 1, wherein the remote control unit comprises:
a signal receiver for receiving an image signal and Hall sensor signals transmitted from the wireless transmission circuit of the capsule in the human body, the image being obtained by the camera;
a joystick for outputting a command signal controlling the robot driving motors for controlling speed and displacement of the cartesian coordinate robot, a command signal controlling the joint driving motor for controlling rotation angle of the 2-DOF rotary joint unit, and a command signal controlling the bed driving motors for controlling angle of the bed by using a bed adjustment switch, according to an operators operation;
a main controller for receiving the image signal from the signal receiver, for displaying the image on a screen, for generating a driving motor control signal for the artesian coordinate robot and 2-DOF rotary joint unit by combining the command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic forces applied to the capsule constant by analyzing the Hall sensor signals of the capsule, for calculating a distance between a surface of the human body and the capsule using the Hall sensor signals and a distance obtained by the distance sensor, and for displaying the calculated distance on the screen;
a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body;
a 2-DOF joint unit controller for controlling the 2-DOF joint unit to adjust a rotation angle of the 2-DOF joint unit according to the driving motor control signal outputted from the main controller or outputted as a result of manual operations to rotate the external permanent magnet in at least two directions, thereby making the capsule in the human body roll, yaw or pitch; and
a bed rotation controller for driving a bed driving motor provided in the bed according to the signal that controls the bed's angle to make the bed roll around a longitudinal axis of the bed, the signal being outputted from the bed adjustment switch provided in the joystick.

7. The control system according to claim 6, wherein the main controller recognizes a shape change of the digestive organs using a frame grabber function from the image obtained by the camera, determines and estimates a forward direction of the capsule in the human body using the camera image or the signals of the Hall sensors provided in the capsule, and displays a position and a path of the capsule in the human body against a fixed coordinate outside the human body by considering the image signal and Hall sensor signals transmitted from the capsule, the position of the capsule against the fixed coordinate, rotation angle of the external permanent magnet, a distance between the capsule and the external permanent magnet, and the estimated direction of the capsule.

8. The control system according to claim 6, wherein the main controller estimates the distance between the external permanent magnet and the capsule by analyzing the Hall sensor signals, measures the distance between the external permanent magnet and the body surface using the distance sensor and thus calculates the distance from the body surface to the capsule.

9. The control system according to claim 6, wherein the main controller further comprises:
a robot control signal outputting unit for outputting a control signal to control speed of the cartesian coordinate robot in X and Y axes directions by combining the command signal controlling the robot driving motors, direction of the capsule and coordinate of the capsule, the command signal controlling speed of the cartesian coordinate robot in X and Y axes directions , and outputting a control signal to control speed and displacement of the cartesian coordinate robot in the Z axis direction by using magnetic force information obtained by combining the command signal controlling the robot driving motors, measured magnetic force of the capsule and reference input value of magnetic force, the command signal controlling speed and displacement of the cartesian coordinate robot in the Z axis direction; and
a direction determining and coordinate calculating unit for determining direction of the capsule by analyzing the two Hall sensor signals transmitted from the signal receiver and the information of shape change recognized by a frame grabber function unit, calculating the coordinate value of the capsule and transmitting the coordinate value to the robot control signal outputting unit and 2-DOF joint unit controller.

10. The control system according to claim 6, wherein the main controller further comprises:
a magnetic force measuring unit for measuring a magnetic force applied to the capsule by analyzing the Hall sensor signals transmitted from the signal receiver and for transmitting the measured value of the magnetic force to the robot control signal outputting unit;
a permanent magnet distance estimating unit for estimating a distance between the permanent magnets of the capsule and the external permanent magnet by analyzing the Hall sensor signals transmitted from the signal receiver; and
a capsule depth calculating unit for calculating a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.

11. The control system according to claim 1, wherein the camera is a CCD camera.

12. The control system according to claim 1, wherein the distance sensor is a photoelectric sensor or ultrasonic sensor.

13. A capsule type endoscope control system for diagnosing digestive organs in a human body comprising:
a medical capsule equipped with at least one permanent magnet, Hall sensors and a camera to diagnose the digestive organs, comprising a wireless transmission circuit for transmitting a series of signals to outside of the body;
a multi-degree of freedom (DOF) rotary joint unit for rotating an external permanent magnet in at least two directions, the external permanent magnet applying magnetic forces to the at least one permanent magnets provided in the capsule;
a distance sensor attached to a lower end of the multi-DOF rotary joint unit, for measuring a distance between the external permanent magnet and a surface of the human body;
a cartesian coordinate robot for moving the external permanent magnet and the multi-DOF rotary joint unit;
a bed for supporting the human body, the bed being able to roll within a certain degree; and
a remote control unit outside the human body for controlling operations of the multi-DOF rotary joint unit, the bed and the cartesian coordinate robot, thereby moving to any position, rotating or stopping the capsule in the human body.

14. A capsule type endoscope control system for diagnosing and/or treating digestive organs in a human body comprising:
a medical capsule equipped with at least one permanent magnet, Hall sensors, a medicine supplying unit and a camera to diagnose and/or treat the digestive organs, comprising a wireless transmission circuit for transmitting a series of signals to outside of the body;
a multi-degree of freedom (DOF) rotary joint unit for rotating an external permanent magnet in at least two directions, the external permanent magnet applying magnetic forces to the at least one permanent magnets provided in the capsule;
a distance sensor attached to a lower end of the multi-DOF rotary joint unit, for measuring a distance between the external permanent magnet and a surface of the human body;
a cartesian coordinate robot for moving the external permanent magnet and the multi-DOF rotary joint unit;
a bed for supporting the human body, the bed being able to roll within a certain degree; and
a remote control unit outside the human body for controlling operations of the multi-DOF rotary joint unit, the bed and the cartesian coordinate robot, thereby moving to any position, rotating or stopping the capsule in the human body.

15. The control system according to claim 2, wherein the remote control unit comprises:
a signal receiver for receiving an image signal and Hall sensor signals transmitted from the wireless transmission circuit of the capsule in the human body, the image being obtained by the camera;
a joystick for outputting a command signal controlling the robot driving motors for controlling speed and displacement of the cartesian coordinate robot, a command signal controlling the joint driving motor for controlling rotation angle of the 2-DOF rotary joint unit, and a command signal controlling the bed driving motors for controlling angle of the bed by using a bed adjustment switch, according to an operator's operation;
a main controller for receiving the image signal from the signal receiver, for displaying the image on a screen, for generating a driving motor control signal for the cartesian coordinate robot and 2-DOF rotary joint unit by combining the command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic force applied to the capsule constant by analyzing the Hall sensor signals of the capsule, for calculating a distance between a surface of the human body and the capsule using the Hall sensor signals and a distance obtained by the distance sensor, and for displaying the calculated distance on the screen;
a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body;
a 2-DOF joint unit controller for controlling the 2-DOF joint unit to adjust rotation angle of the 2-DOF joint unit according to the driving motor control signal outputted from the main controller or outputted as a result of manual operations to rotate the external permanent magnet in at least two directions, thereby making the capsule in the human body roll, yaw or pitch; and
a bed rotation controller for driving a bed driving motor provided in the bed according to the signal that controls the bed's angle to make the bed roll around longitudinal axis of the bed, the signal being outputted from the bed adjustment switch provided in the joystick.

16. The control system according to claim 15, wherein the main controller recognizes a shape change of the digestive organs using a frame grabber function from the image obtained by the camera, determines and estimates a forward direction of the capsule in the human body using the camera image or the signals of the Hall sensors provided in the capsule, and displays a position and a path of the capsule in the human body against a fixed coordinate outside the human body by considering the image signal and Hall sensor signals transmitted from the capsule, the position of the capsule against the fixed coordinate, rotation angle of the external permanent magnet, a distance between the capsule and the external permanent magnet, and the estimated direction of the capsule.

17. The control system according to claim 15, wherein the main controller estimates the distance between the external permanent magnet and the capsule by analyzing the Hall sensor signals, measures the distance between the external permanent magnet and the body surface using the distance sensor and thus calculates the distance from the body surface to the capsule.

18. The control system according to claim 15, wherein the main controller further comprises:
a robot control signal outputting unit for outputting a control signal to control a speed of the cartesian coordinate robot in X and Y axes directions by combining the command signal controlling the robot driving motors, a direction of the capsule and a coordinate of the capsule, the command signal controlling speed of the cartesian coordinate robot in X and Y axes directions, and outputting a control signal to control a speed and displacement of the cartesian coordinate robot in Z axis direction by using magnetic force information obtained by combining the command signal controlling the robot driving motors, measured magnetic force of the capsule and reference input value of magnetic force, the command signal controlling speed and displacement of the cartesian coordinate robot in Z axis direction; and
a direction determining and coordinate calculating unit for determining direction of the capsule by analyzing the Hall sensor signals transmitted from the signal receiver and information of shape change recognized by a frame grabber function unit, calculating the coordinate value of the capsule and transmitting the coordinate value to the robot control signal outputting unit and 2-DOF joint unit controller.

19. The control system according to claim 15, wherein the main controller further comprises:
a magnetic force measuring unit for measuring a magnetic force applied to the capsule by analyzing the Hall sensor signals transmitted from the signal receiver and for transmitting the measured value of the magnetic force to the robot control signal outputting unit;
a permanent magnet distance estimating unit for estimating a distance between the permanent magnets of the capsule and the external permanent magnet by analyzing the Hall sensor signals transmitted from the signal receiver; and
a capsule depth calculating unit for calculating a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.

20. The control system according to claim 3, wherein the remote control unit comprises:
a signal receiver for receiving an image signal and Hall sensor signals transmitted from the wireless transmission circuit of the capsule in the human body, the image being obtained by the camera;
a joystick for outputting a command signal controlling the robot driving motors for controlling speed and displacement of the cartesian coordinate robot, a command signal controlling the joint driving motor for controlling rotation angle of the 2-DOF rotary joint unit, and a command signal controlling the bed driving motors for controlling an angle of the bed by using a bed adjustment switch, according to an operator's operation;
a main controller for receiving the image signal from the signal receiver, for displaying the image on a screen, for generating a driving motor control signal for the cartesian coordinate robot and 2-DOF rotary joint unit by combining command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic force applied to the capsule constant by analyzing the Hall sensor signals of the capsule, for calculating a distance between a surface of the human body and the capsule using the Hall sensor signals and a distance obtained by the distance sensor, and for displaying the calculated distance on the screen;
a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body;
a 2-DOF joint unit controller for controlling the 2-DOF joint unit to adjust a rotation angle of the 2-DOF joint unit according to the driving motor control signal outputted from the main controller or outputted as a result of manual operations to rotate the external permanent magnet in at least two directions, thereby making the capsule in the human body roll, yaw or pitch; and
a bed rotation controller for driving a bed driving motor provided in the bed according to the signal that controls the bed's angle to make the bed roll around a longitudinal axis of the bed, the signal being outputted from the bed adjustment switch provided in the joystick.

21. The control system according to claim 20, wherein the main controller recognizes a shape change of the digestive organs using a frame grabber function from the image obtained by the camera, determines and estimates a forward direction of the capsule in the human body using the camera image or the signals of the two Hall sensors provided in the capsule, and displays a position and a path of the capsule in the human body against a fixed coordinate outside the human body by considering the image signal and Hall sensor signals transmitted from the capsule, a position of the capsule against the fixed coordinate, a rotation angle of the external permanent magnet, a distance between the capsule and the external permanent magnet, and the estimated direction of the capsule.

22. The control system according to claim 20, wherein the main controller estimates the distance between the external permanent magnet and the capsule by analyzing the Hall sensor signals, measures the distance between the external permanent magnet and the body surface using the distance sensor and thus calculates the distance from the body surface to the capsule.

23. The control system according to claim 20, wherein the main controller further comprises:
a robot control signal outputting unit for outputting control signal to control speed of the cartesian coordinate robot in X and Y axes directions by combining the command signal controlling the robot driving motors, direction of the capsule and coordinate of the capsule, the command signal controlling speed of the cartesian coordinate robot in X and Y axes direction, and outputting control signal to control speed and displacement of the cartesian coordinate robot in the Z axis direction by using magnetic force information obtained by combining the command signal controlling the robot driving motors, measured magnetic force of the capsule and reference input value of magnetic force, the command signal controlling speed and displacement of the cartesian coordinate robot in the Z axis direction; and
a direction determining and coordinate calculating unit for determining direction of the capsule by analyzing the Hall sensor signals transmitted from the signal receiver and the information of shape change recognized by a frame grabber function unit, calculating the coordinate value of the capsule and transmitting the coordinate value to the robot control signal outputting unit and 2-DOF joint unit controller.

24. The control system according to claim 20, wherein the main controller further comprises:
a magnetic force measuring unit for measuring a magnetic force applied to the capsule by analyzing the Hall sensor signals transmitted from the signal receiver and for transmitting the measured value of the magnetic force to the robot control signal outputting unit;
a permanent magnet distance estimating unit for estimating a distance between the permanent magnets of the capsule and the external permanent magnet by analyzing the Hall sensor signals transmitted from the signal receiver; and
a capsule depth calculating unit for calculating a distance from the body surface to the capsule with the distance, between the at least one permanent magnet of the capsule and the external permanent magnet, estimated by the permanent magnet distance estimating unit and the distance, between the external permanent magnet and the body surface, obtained by the distance sensor.

25. The control system according to claim 4, wherein the remote control unit comprises:
a signal receiver for receiving an image signal and Hall sensor signals transmitted from the wireless transmission circuit of the capsule in the human body, the image being obtained by the camera;
a joystick for outputting a command signal controlling the robot driving motors for controlling speed and displacement of the cartesian coordinate robot, a command signal controlling the joint driving motor for controlling rotation angle of the 2-DOF rotary joint unit, and a command signal controlling the bed driving motors for controlling an angle of the bed by using a bed adjustment switch, according to an operator's operation;
a main controller for receiving the image signal from the signal receiver, for displaying the image on a screen, for generating a driving motor control signal for the cartesian coordinate robot and 2-DOF rotary joint unit by combining command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic force applied to the capsule constant by analyzing the Hall sensor signals of the capsule, for calculating a distance between a surface of the human body and the capsule using the Hall sensor signals and a distance obtained by the distance sensor, and for displaying the calculated distance on the screen;
a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body;
a 2-DOF joint unit controller for controlling the 2-DOF joint unit to adjust rotation angle of the 2-DOF joint unit according to the driving motor control signal outputted from the main controller or outputted as a result of manual operations to rotate the external permanent magnet in at least two directions, thereby making the capsule in the human body roll, yaw or pitch; and
a bed rotation controller for driving a bed driving motor provided in the bed according to the signal that controls the bed's angle to make the bed roll around longitudinal axis of the bed, the signal being outputted from the bed adjustment switch provided in the joystick.

26. The control system according to claim 25, wherein the main controller recognizes a shape change of the digestive organs using a frame grabber function from the image obtained by the camera, determines and estimates a forward direction of the capsule in the human body using the camera image or the signals of the Hall sensors provided in the capsule, and displays a position and a path of the capsule in the human body against a fixed coordinate outside the human body by considering the image signal and Hall sensor signals transmitted from the capsule, a position of the capsule against the fixed coordinate, a rotation angle of the external permanent magnet, a distance between the capsule and the external permanent magnet, and the estimated direction of the capsule.

27. The control system according to claim 25, wherein the main controller estimates the distance between the external permanent magnet and the capsule by analyzing the Hall sensor signals, measures the distance between the external permanent magnet and the body surface using the distance sensor and thus calculates the distance from the body surface to the capsule.

28. The control system according to claim 25, wherein the main controller further comprises:
a robot control signal outputting unit for outputting control signal to control speed of the cartesian coordinate robot in X and Y axes directions by combining the command signal controlling the robot driving motors, direction of the capsule and coordinate of the capsule, the command signal controlling speed of the cartesian coordinate robot in X and Y axes directions, and outputting a control signal to control speed and displacement of the cartesian coordinate robot in Z axis direction by using magnetic force information obtained by combining the command signal controlling the robot driving motors, a measured magnetic force of the capsule and a reference input value of magnetic force, the command signal controlling speed and displacement of the cartesian coordinate robot in the Z axis direction; and
a direction determining and coordinate calculating unit for determining direction of the capsule by analyzing the Hall sensor signals transmitted from the signal receiver and information of shape change recognized by a frame grabber function unit, calculating a coordinate value of the capsule and transmitting the coordinate value to the robot control signal outputting unit and 2-DOF joint unit controller.

29. The control system according to claim 25, wherein the main controller further comprises:
a magnetic force measuring unit for measuring a magnetic force applied to the capsule by analyzing the Hall sensor signals transmitted from the signal receiver and for transmitting the measured value of the magnetic force to the robot control signal outputting unit;
a permanent magnet distance estimating unit for estimating a distance between the permanent magnets of the capsule and the external permanent magnet by analyzing the Hall sensor signals transmitted from the signal receiver; and
a capsule depth calculating unit for calculating a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.

30. The control system according to claim 5, wherein the remote control unit comprises:
a signal receiver for receiving an image signal and Hall sensor signals transmitted from the wireless transmission circuit of the capsule in the human body, the image being obtained by the camera;
a joystick for outputting a command signal controlling the robot driving motors for controlling speed and displacement of the cartesian coordinate robot, a command signal controlling the joint driving motor for controlling a rotation angle of the 2-DOF rotary joint unit, and a command signal controlling the bed driving motors for controlling angle of the bed by using a bed adjustment switch, according to an operator's operation;
a main controller for receiving the image signal from the signal receiver, for displaying the image on a screen, for generating driving motor control signal for the cartesian coordinate robot and 2-DOF rotary joint unit by combining the command signals outputted from the joystick and a stick-slip preventing operation, for outputting the driving motor control signal to corresponding controllers, for controlling a Z-axis driving motor to adjust speed and displacement of the cartesian coordinate robot in a Z-axis direction to keep the magnetic force applied to the capsule constant by analyzing the Hall sensor signals of the capsule, for calculating a distance between a surface of the human body and the capsule using the Hall sensor signals and a distance obtained by the distance sensor, and for displaying the calculated distance on the screen;
a robot controller for controlling X and Y axes driving motors of the cartesian coordinate robot to adjust speed of the cartesian coordinate robot and controlling the Z axis driving motor to adjust speed and displacement of the cartesian coordinate robot, according to the driving motor control signal for the cartesian coordinate robot, to move the external permanent magnet in a transverse direction, a longitudinal direction and a vertical direction of the human body to move the capsule in the human body;
a 2-DOF joint unit controller for controlling the 2-DOF joint unit to adjust rotation angle of the 2-DOF joint unit according to the driving motor control signal outputted from the main controller or outputted as a result of manual operations to rotate the external permanent magnet in at least two directions, thereby making the capsule in the human body roll, yaw or pitch; and
a bed rotation controller for driving a bed driving motor provided in the bed according to the signal that controls the bed angle to make the bed roll around longitudinal axis of the bed, the signal being outputted from the bed adjustment switch provided in the joystick.

31. The control system according to claim 30, wherein the main controller recognizes a shape change of the digestive organs using a frame grabber function from the image obtained by the camera, determines and estimates a forward direction of the capsule in the human body using the camera image or the signals of the two Hall sensors provided in the capsule, and displays a position and a path of the capsule in the human body against a fixed coordinate outside the human body by considering the image signal and Hall sensor signals transmitted from the capsule, the position of the capsule against the fixed coordinate, a rotation angle of the external permanent magnet, a distance between the capsule and the external permanent magnet, and the estimated direction of the capsule.

32. The control system according to claim 30, wherein the main controller estimates the distance between the external permanent magnet and the capsule by analyzing the Hall sensor signals, measures the distance between the external permanent magnet and the body surface using the distance sensor and thus calculates the distance from the body surface to the capsule.

33. The control system according to claim 30, wherein the main controller further comprises:
a robot control signal outputting unit for outputting a control signal to control speed of the cartesian coordinate robot in X and Y axes direction by combining the command signal controlling the robot driving motors, a direction of the capsule and a coordinate of the capsule, the command signal controlling speed of the cartesian coordinate robot in X and Y axes direction, and outputting a control signal to control speed and displacement of the cartesian coordinate robot in Z axis direction by using magnetic force information obtained by combining the command signal controlling the robot driving motors, measured magnetic force of the capsule and a reference input value of magnetic force, the command signal controlling speed and displacement of the cartesian coordinate robot in Z axis direction; and
a direction determining and coordinate calculating unit for determining direction of the capsule by analyzing the two Hall sensor signals transmitted from the signal receiver and information of shape change recognized by a frame grabber function unit, calculating the coordinate value of the capsule and transmitting the coordinate value to the robot control signal outputting unit and 2-DOF joint unit controller.

34. The control system according to claim 30, wherein the main controller further comprises:
a magnetic force measuring unit for measuring a magnetic force applied to the capsule by analyzing the Hall sensor signals transmitted from the signal receiver and for transmitting the measured value of the magnetic force to the robot control signal outputting unit;
a permanent magnet distance estimating unit for estimating a distance between the permanent magnets of the capsule and the external permanent magnet by analyzing the Hall sensor signals transmitted from the signal receiver; and
a capsule depth calculating unit for calculating a distance from the body surface to the capsule with the distance, between the permanent magnets of the capsule and the external permanent magnet, estimated by the permanent magnet distance estimating unit and the distance, between the external permanent magnet and the body surface, obtained by the distance sensor.
